(19) United States
(12) Patent Application Publication
(10) Pub. No.: US 2025/0223244 A1
(43) Pub. Date: Jul. 10, 2025

(54) DEHYDROGENATION METHODS

(71) Applicant: The Trustees of Boston College, Chestnut Hill, MA (US)

(72) Inventors: Dunwei Wang, Newton, MA (US); Yucheng Yuan, Brighton, MA (US); Yuhan Zhang, Brighton, MA (US)

(21) Appl. No.: 19/015,367

(22) Filed: Jan. 9, 2025

Related U.S. Application Data
(60) Provisional application No. 63/619,690, filed on Jan. 10, 2024.

(51) Int. Cl. C07C 5/32; B01J 31/02

(52) U.S. Cl. CPC C07C 5/322; B01J 31/0268; B01J 2231/76; C07C 2523/75; C07C 2523/755; C07C 2531/34

(57) ABSTRACT

Disclosed is a method of preparing propylene including the step of irradiating propane in the presence of a catalyst containing a decatungstate salt and a co-catalyst, in which the co-catalyst has cobalt or nickel. Also provided are catalysts and methods of dehydrogenating alkanes.

### CROSS REFERENCE TO RELATED APPLICATION

The application claims the benefit of priority to U.S. Application Ser. No. 63/619,690 filed on Jan. 10, 2024, the entire content of which is incorporated by reference.

### GOVERNMENT SUPPORT

The invention was made with government support under grant number CHE 1955098 awarded by the National Science Foundation. The government has certain rights in the invention.

### FIELD OF INVENTION

The disclosure relates to dehydrogenation reactions using photocatalysts for preparing alkenes.

### BACKGROUND

Propylene is an important feeding stock for the synthesis of commodity chemicals such as polypropylene and acrylonitrile. The demand for propylene has outpaced the supply, which is provided mainly by the refinery industry.

Propane dehydrogenation (PDH) offers a promising production route, benefiting from not only the abundance of propane supplies from the shale gas but also potential high selectivity of propane to propylene conversion. The thermodynamic limit of conventional PDH processes is recognized as a key factor that limits propylene production through this route. See Hannagan et al., Science 372, 1444-1447 (2021); Zhou et al., Nat. Catal. 5, 1145-1156 (2022); Nakaya et al., Nat. Commun. 11, 2838 (2020); Sun et al., Angew. Chem. 132, 19618-19627 (2020); and Ma et al., Nat. Catal. 6, 506-518 (2023).

PDH is an endothermic reaction. Under conventional processes, higher yields of propylene are expected at higher temperatures. Nevertheless, a high temperature reduces the conversion selectivity and catalyst durability. See Chen et al., Chem. Soc. Rev. 50, 3315-3354 (2021); and Zeng et al., ChemCatChem 15, e202201405 (2023). Consequently, most conventional PDH productions are operated at temperatures ranging from 550 to 620° C. See Chen et al., Mol. Catal. 476, 110508 (2019). In addition, as an entropy-increasing reaction, PDH is favored at a low partial propane pressure, which is achieved by diluting propane with an inert gas, therefore inevitably decreasing propylene yields and adding separation costs. The most recent PDH process operating at the atmospheric pressure and 580° C. has obtained only a 40% conversion rate. See Motagamwala et al., Science 373, 217-222 (2021).

There is a need to develop a process having a high conversion of propane to propylene under mild conditions in term of temperature and pressure.

### SUMMARY

This invention is based on an unexpected discovery of a process that converts propane to propylene at high conversion rates under mild conditions using decatungstate catalysts.

Accordingly, one aspect of this invention relates to a method of preparing propylene comprising the step of irradiating propane in the presence of a catalyst containing a decatungstate salt and a co-catalyst containing cobalt or nickel.

The method can have one or more of the following preferred features in any combinations.
(i) The decatungstate salt is sodium decatungstate (NaDT) or tetraphenyl-phosphonium decatungstate (TPPDT).
(ii) The decatungstate salt is NaDT present at a level of 0.001 mol % to 30 mol % relative to propane, preferably at 1 mol % to 9 mol %, and more preferably at 2.8 mol % to 3.2 mol %.
(iii) The co-catalyst is cobaloxime pyridine chloride (COPC), Co(dmgH)₂, N,N′,N″,N′″-(tetrafluorodiborato)bis[μ-(2,3-butanedionedioximato)]-cobalt(II) dihydrate, nickel(II) trifluoromethanesulfonate, nickel(II) tetrafluoroborate, vitamin B₁₂, cobaloximes with halides, substituted pyridine ligands, 4-methoxypyridine, isonicotinonitrile, isonicotinic acid, 4-methylpyridine, or imidazoles.
(iv) The co-catalyst is COPC present at a level of 0.001 mol % to 20 mol % relative to propane, preferably at 0.1 mol % to 6 mol %, and more preferably at 0.5 mol % to 3.2 mol %.
(v) The catalyst further contains dimethylglyoxime at a level of 0.01 mol % to 20 mol % relative to propane, preferably at 0.1 mol % to 12 mol %, and more preferably at 6 mol % to 9 mol %.
(vi) The irradiating step is performed in a solvent selected from the group consisting of acetonitrile, acetone, benzonitrile, acetonitrile-water, and chlorobenzene.
(vii) The irradiating step is performed absent of a solvent.
(viii) The irradiating step is performed at a temperature of −40° C. to 80° C., preferably at 0° C. to 40° C., and more preferably at 13° C. to 25° C.
(ix) The irradiating step is performed at a pressure of 0.001 bars to 14 bars, preferably at 0.1 bars to 7 bars, and more preferably at 0.2 bars to 3 bars.
(x) The irradiating step utilizes a light having a wavelength of 200 nm to 410 nm, preferably 250 nm to 390 nm, and more preferably 300 nm to 370 nm.
(xi) The irradiation step is performed for 1 minute to 50 hours, preferably for 1 hour to 20 hours, and more preferably from 3 hours to 16 hours.
(xii) The irradiation step is performed in a batch reactor or a flow reactor.

Another aspect of this invention relates to a catalyst comprising a decatungstate salt and a co-catalyst, in which the co-catalyst is cobaloxime pyridine chloride. Preferably, the decatungstate salt is sodium decatungstate (NaDT) or tetraphenylphosphonium decatungstate (TPPDT),

Also within the scope of this invention is a catalyst comprising TPPDT. The TPPDT catalyst can further contain cobalt, nickel, or dimethylglyoxime as described above.

Still within the scope of this invention is a method of dehydrogenating an alkane to prepare an alkene, including the step of irradiating the alkane in the presence of a catalyst described above. Suitable alkanes contain 2 to 20 carbon atoms such as ethane, propane, butane, pentane, hexane, heptane, octane, nonane, decane, cyclopentane, cyclohexane, cycloheptane, cyclooctane, cyclononane, and cyclodecane.

The details of the invention are set forth in the definitions and the detailed description below. Other features, objects, and advantages of the invention will be apparent from the following actual examples and claims.

### DETAILED DESCRIPTION

A catalyst of this invention includes a decatungstate salt such as sodium decatungstate (NaDT), tetrabutylammonium decatungstate (TBADT), or tetraphenylphosphonium decatungstate (TPPDT). In a preferred embodiment, the catalyst has a co-catalyst containing cobalt or nickel. Optionally, the catalyst contains a ligand such as dimethylglyoxime.

The catalyst of this invention can be a single catalyst (e.g., TPPDT) or a cooperative photocatalyst system (e.g., NaDT+COPC). Both are useful as photocatalysts in preparing propylene by dehydrogenating propane under irradiation.

### Decatungstate Salts

NaDT is represented by formula Na₄[W₁₀O₃₂]. An exemplary preparation method is described in Sarver et al., J. Am. Chem. Soc. 143, 9737-9743 (2021).

A preparation method of TBADT is illustrated in Protti et al., Chem. Commun. 7351-7353 (2009) doi: 10.1039/B917732A.

The decatungstate salt contains a cation such as an alkali or alkaline earth metal ion selected from lithium, sodium, potassium, rubidium, caesium, calcium, magnesium, manganese, barium, copper, zinc, aluminum, and ferric. Alternatively, the decatungstate salt contains an organic cation. One class of the organic cations are those derived from organic amines. Examples include tetrabutylammonium, methyl ammonium (CH₃NH₃⁺), ethyl ammonium (CH₃CH₂NH₃)⁺, formamidinium (CH(NH₂)₂⁺), methylformamidinium (CH₃C(NH₂)₂⁺), guanidium C((NH)₂)₃⁺), tetramethylammonium ((CH₃)₄N⁺), dimethyl-ammonium, diethylammonium, trimethylammonium ((CH₃)₃NH⁺), triethylammonium, tributylammonium, diethylmethylammonium, hydroxyethylammonium, methoxymethyl-ammonium, dibutylammonium, methylbutylammonium, anilinium, pyridinium, 2-methyl-pyridinium, imidazolium, 1-methylimidazolium, 1,2-dimethylimidazolium, imidazolinium, 1-ethylimidazolium, 1-(4-sulfobutyl)-3-methylimidazolium, 1-allylimidazolium, quinolinium, isoquinolinium, pyrrolinium, pyrrolininium and pyrrolidinium. Examples of ammonium ions also include aprotic ions such as 1-butyl-1-methylpyrrolidinium, tetramethylammonium, tetraethylammonium, tetra-n-butylammonium, n-butyl-triethylammonium, benzyl-tri-methylammonium, tri-n-butylmethylammonium, benzyl-tri-ethylammonium, 1-methyl-pyridinium, 1-butyl-3,5-dimethylpyridinium, 1,2,4-trimethylpyrazolium, trimethylhydroxy-ethylammonium (choline), tri-(hydroxyethyl)methylammonium, dimethyl-di(polyoxy-ethylene)ammonium, 1,2,3-trimethylimidazolium, 1-butyl-3-methylimidazolium, 1-ethyl-2,3-dimethylimidazolium, 1-allyl-3-methylimidazolium, 1-hydroxyethyl-3-methylimidazolium, 1,3-dimethylimidazolium, 1-ethyl-1-methylpiperidinium, 4-ethyl-4-methylmorpholinium, 1-(cyanomethyl)-3-methylimidazolium, 1-(3-cyanopropyl)pyridinium, 1,3-bis(cyanomethyl)-imidazolium, 1-hexyl-3-methylimidazolium and 1-ethyl-3-methylimidazolium.

Another class of organic cations are phosphoniums such as tetraphenyl-phosphonium, methyltriphenylphosphonium, tetrabutylphosphonium, tributylmethyl-phosphonium, triethylmethylphosphonium, trihexyltetradecylphosphonium, triphenylpropyl-phosphonium and tetrakis(hydroxymethyl)phosphonium.

Additional cations include ammonium (NH₄⁺), 1-pyrrolininium, 2-pyrrolininium, 3-pyrrolininium,

### Co-catalysts

Suitable co-catalysts contain cobalt or nickel including their salts. Exemplary co-catalysts are cobaloxime pyridine chloride (COPC), Co(dmgH)₂, N,N′,N″,N′″-(tetrafluorodiborato)-bis[μ-(2,3-butanedionedioximato)]-cobalt(II) dihydrate, nickel(II) trifluoromethanesulfonate, and nickel(II) tetrafluoroborate. Additional co-catalysts include vitamin B₁₂, cobaloximes with halides, substituted pyridine ligands, 4-methoxypyridine, isonicotinonitrile, isonicotinic acid, 4-methylpyridine, and imidazoles.

### Ligands

The catalysts of this invention can further contain a ligand to improve efficiency. Suitable ligands include dimethylglyoxime, glyoxime, diphenylglyoxime, 1,2-cyclohexane-dione dioxime, dichloroglyoxime, pyridine, methoxypyridines, cyanopyridines, pyridine-carboxylic acids, alkylpyridines, halopyridines, 4-dimethylaminopyridine, imidazole, 3,4-dihydro-2H-pyrroles, and benzimidazoles.

Certain terminology is used in the following description for convenience only and is not limiting.

A range expressed as being between two numerical values, one as a low endpoint and the other as a high endpoint, includes the values between the numerical values and the low and high endpoints. Embodiments herein include subranges of a range herein, where the subrange includes a low and high endpoint of the subrange selected from any increment within the range selected from each single increment of the smallest significant figure, with the condition that the high endpoint of the subrange is higher than the low endpoint of the subrange.

Further embodiments herein include replacing one or more “including” or “comprising” in an embodiment with “consisting essentially of” or “consisting of.” “Including” and “comprising,” as used herein, are open ended, include the elements recited, and do not exclude the addition of one or more other element. “Consisting essentially of” means that addition of one or more element compared to what is recited is within the scope, but the addition does not materially affect the basic and novel characteristics of the combination of explicitly recited elements. “Consisting of” refers to the recited elements, but excludes any element, step, or ingredient not specified.

The words “a” and “one,” as used in the claims and in the corresponding portions of the specification, are defined as including one or more of the referenced items unless specifically stated otherwise. This terminology includes the words above specifically mentioned, derivatives thereof, and words of similar import. The phrase “at least one” followed by a list of two or more items, such as “A, B, or C” or “A, B, and C” means any individual one of A, B or C as well as any combination thereof.

The term “alkane” herein refers to a straight, branched, or cyclic hydrocarbon compound, containing 1-20 (e.g., 1-10 and 1-6) carbon atoms. Examples include ethane, n-propane, i-propane, n-butane, i-butane, t-butane, and cyclohexane.

The term “alkene” herein refers to a straight, branched, or cyclic hydrocarbon compound having at least a double bond and containing 2-20 (e.g., 2-10 and 2-6) carbon atoms. Examples include ethylene, n-propylene, i-propylene, n-butylene, i-butylene, t-butylene, and cyclohexene.

The method of this invention dehydrogenates propane to produce propylene at a high conversion rate (e.g., greater than 45%, greater than 50%, greater than 60%, greater than 70%, greater than 80%, greater than 90%, and greater than 95%) under ambient conditions (e.g., room temperature and one atmospheric pressure) through photocatalysis. In addition, the method achieves near-unity selectivity to propylene (e.g., a selectivity greater than 70%, 80%, 90%, 95%, 98%, and 99%).

UV light is applied (e.g., λ=365 nm; Power: 485 mW/cm²) as an energy input to facilitate the PDH reaction. A skilled person in the art would be able to determine the light source, wavelength, and energy output taking into consideration of catalyst, substrate, scale, solvent, etc.

Preferably, the catalyst of this invention is a cooperative photocatalyst system containing a decatungstate salt (e.g., NaDT, (Na₄[W₁₀O₃₂]) and a co-catalyst (e.g., cobaloxime pyridine chloride COPC). Not to be bound by any theory, NaDT is effective to activate the C—H bond of propane and abstract one hydrogen atom upon photoexcitation, producing an intermediate, i.e., a propyl radical. In turn, NaDT is reduced. The intermediate is then further activated by COPC to abstract a second hydrogen in the a position, leading to the formation of propylene. COPC is oxidized in the second step. The reduced DT and oxidized COPC finally react with each other to release H₂ and regenerate both. The net overall reaction is PDH with the production of propylene and H₂. In a typical reaction, 1 atm of propane is introduced into a vacuumed reactor containing, relative to propane, NaDT (0.001-30 mol %, 1-9 mol %, 2.8-3.2 mol %, 2 mol %, 2.5 mol %, 3 mol %, 3.5 mol %, and 4 mol %), COPC (0.001-20 mol %, 0.1-6 mol %, 0.5-3.2 mol %, 0.8 mol %, 1 mol %, 1.5 mol %, 2 mol %, 2.5 mol %, and 3 mol %), and dimethylglyoxime (dmgH₂, 0.01-20 mol %, 0.1-12 mol %, 6-9 mol %, 2 mol %, 3 mol %, 4 mol %, 5 mol %, 6 mol %, 7 mol %, and 8 mol %). All components are preferably dissolved in a solvent such as acetonitrile. The resulting mixture is then irradiated with a light (e.g., 485 mW/cm² of 365 nm) for a period of time (e.g., 0.5-48 hours, 1-40 hours, 2-35 hours, 5-30 hours, 10-25 hours, 12 hours, 15 hours, 20 hours, and 24 hours) at an ambient temperature (e.g., 22° C., 0° C., 10° C., 22° C., 30° C., 40° C., and 50° C.).

The molar ratio between NaDT and COPC is typically in the range of 1:10 to 10:1 (e.g., 1:5 to 5:1; 1:3 to 3:1; 1:2 to 2:1, 1:1, and 3:2).

A co-catalyst (e.g., COPC) can greatly increase the conversion rate as compared to the decatungstate salt (e.g., NaDT) alone. It is believed that the advantages offered by COPC are twofold. First, as a mild hydrogen atom transfer (HAT) catalyst, it is not expected to compete with DT in activating propane, which otherwise would undermine the effectiveness of DT and consume COPC. Instead, COPC only reacts with the propyl radical as the product of DT hydrogen atom abstraction (HAA) of propane. Second, the product of HAA by COPC is a hydride that readily reacts with H⁺[W₁₀O₃₂]⁵⁻ to release H₂ and regenerate both catalysts, as evidenced by stoichiometric release of H₂ relative to propylene. Cooperatively, a catalytic cycle is complete to regenerate both catalysts during the PDH process.

Other suitable co-catalysts include cobalt based catalysts such as in situ generated [Co(dmgH)₂] species (formed by combining cobalt salt with dmgH₂) and N,N′,N″,N″′-(tetra-fluorodiborato)bis[μ-(2,3-butanedionedioximato)]cobalt(II) dihydrate (Co(dmgBF₂)₂(H₂O)₂, COBF), and nickel based catalysts such as nickel(II) trifluoromethanesulfonate (Ni(Tf)₂) and nickel(II) tetrafluoroborate hexahydrate (Ni(BF₄)₂·6H₂O).

Also envisioned in this invention is a ligand included in the catalyst to stabilize the catalyst as NaDT and COPC might be prone to degradation. An exemplary ligand is dmgH₂. Addition of ligand enhanced PDH conversion, increasing it from 48% to 60% with the addition of 6 mol % of dmgH₂ demonstrated in an example below. When the addition was increased to 9 mol %, the conversion was improved to 61.4%. See Table 1 below. Fresh COPC can be added to the reaction mixture to further improve the conversion rate. It is preferably added after 10 hours, 15 hours, 20 hours, 30 hours, or 40 hours.

The time needed to complete the PDH reaction depends on several factors, e.g., light source, wavelength, light energy output, concentration of the catalyst, concentration of propane, removal of propylene and H₂, temperature, solvent, scale of production, etc. The PDH method using the catalyst of this invention proceeds at a fast pace (e.g., for 0.1 to 6 h) and can complete at 20 h to 48 h.

The reaction achieves a high conversion rate (e.g., 60% and 69% as shown in Table 1 in several examples under the conditions described above) with >99% selectivity to propylene, along with H₂ gas generation at a 1:1 ratio to propylene.

The excellent selectivity of PDH is corroborated by the fact that no measurable byproducts involving propane were detected in Examples described below.

The method of this invention can also be applied to substrates other than propane such as ethane. An ethane dehydrogenation (EDH) using the catalyst of this invention obtained a conversion of 26.8% and >99% ethylene selectivity under standard reaction conditions described above.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever.

All publications, including patent documents, cited herein are incorporated by reference in their entirety.

### EXAMPLES

The following non-limiting examples are provided to illustrate particular embodiments. The embodiments throughout may be supplemented with one or more details from one or more examples below, and/or one or more elements from an embodiment may be substituted with one or more details from one or more examples below.

### Materials and Reagents

Propane (99.5%), propylene (19.18%, balanced with helium), hydrogen (UHP), and argon (UHP) were purchased from Airgas (Radnor, PA). All solvents including acetonitrile, acetone, and benzonitrile were purchased from commercial sources and used without further purification unless otherwise stated. Sodium tungstate dihydrate (Na₂WO₄·2H₂O, 99+%), and tetrabutylammonium bromide (TBAB, 99+%) were purchased from Acros Organics (Belgium). Tetraphenylphosphonium bromide (TPPB, 98%) was purchased from Ambeed (Arlington Heights, IL). N,N′,N″,N′″-(Tetrafluorodiborato)bis[μ-(2,3-butanedionedioximato)]cobalt(II) dihydrate (COBF, >98%) was purchased from Strem (Newburyport, MA). Chloro(pyridine)bis(dimethylglyoximato)cobalt(III) (COPC) and Nickel(II) bis(trifluoromethanesulfonimide) hydrate ((Ni(NTf)₂, 95%) were purchased from Sigma-Aldrich (St. Louis, MO). Dimethylglyoxime (dmgH₂, 99+%) and Nickel(II) tetrafluoroborate hexahydrate ((NiBF₄)₂·6H₂O, 99%) were purchased from Thermo Scientific (Waltham, MA).

Syntheses of DT catalysts

Sodium decatungstate (NaDT) was prepared as follows. Na₂WO₄·2H₂O (22 g, 66.7 mmol) was dissolved in distilled water (125 mL), and the solution was heated to 90° C. with stirring. 125 mL of 1 M HCl boiled at 90° C. was added to the solution, and the resulting solution was heated at 90° C. for 40 seconds and then put into an ice bath while stirring. Solid NaCl (90 g) was added to the solution and was stirred at 0° C. for 1 h. Then the precipitate was filtered and washed subsequently with water (30 mL), ethanol (30 mL), and diethyl ether (30 mL) at 0° C., and was suspended in hot acetonitrile (2×100 mL) while stirring at 90° C. for 30 min. The solution was then centrifuged, and the supernatant was collected in a round-bottomed flask. The solvent was removed by rotary evaporation, and NaDT was collected and dried at 60° C. under vacuum overnight.

Tetrabutylammonium decatungstate (TBADT) was prepared following a similar procedure. TBAB (4.8 g, 14.9 mmol) and Na₂WO₄·2H₂O (10 g, 30.3 mmol) were dissolved in water (50 mL and 100 mL, separately). The solutions were acidified to pH 2 with concentrated hydrochloric acid solution and then heated to 90° C. The solutions were mixed at 90° C., and precipitation was observed immediately, indicating the formation of TBADT. The slurry was stirred for 30 minutes in a 90° C. water bath, then cooled to room temperature, and filtered with a Büchner funnel. The solid phase was washed with DI water and THF (3×30 mL) and dried in a vacuum oven at 90° C. overnight. The crude TBADT was further purified by recrystallization in refluxing DCM (1 g:20 mL) for 2 hours. The mixture was cooled on an ice bath, and then filtered to obtain pure TBADT as a transparent crystal with a light-yellow color.

Tetraphenylphosphonium decatungstate (TPPDT) was prepared via a similar procedure as TBADT. Na₂WO₄·2H₂O (1.67 g, 5 mmol) and TPPB, (0.76 g, 1.8 mmol) was separately dissolved in water (50 mL). The solutions were acidified with concentrated aqueous hydrochloric acid to pH 2, heated to 90° C., and mixed at this temperature. Precipitation was observed immediately, indicating the formation of TPPDT. The resultant slurry was stirred for 30 minutes in a 90° C. water bath, cooled to room temperature, and filtered with a Büchner funnel. The solid phase was washed with water and acetone and dried in a vacuum oven at 60° C. overnight. The crude TPPDT was further purified by refluxing in acetonitrile (1 g:20 mL) for 2 hours. The mixture was cooled on an ice bath, and then filtered to obtain pure TPPDT as a white crystal.

To characterize the DT anions in these catalysts, they were dissolved in acetonitrile at a concentration of 10⁻⁵ M for the UV-vis measurement. The successful syntheses of DT catalysts were confirmed by the characteristic absorption peak at 323 nm in the UV-vis spectra recorded on Agilent Cary 60 UV/VIS Spectrometer.

### Measurements of Products

The partial pressures of propane and propylene in the gas phase were measured by gas chromatography (GC, SRI Instruments®, Multiple Gas Analyzer #5) equipped with a 2-meter HayeSep®-D column and a flame ionization detector (FID), while the partial pressure of hydrogen was measured with a 2-meter Molecular Sieve 5 Å column and a thermal conductivity detector (TCD). Before measurements, the reactor was charged with Ar to 15 bars as indicated by the gauge on the top of the reactor. Then the reactor was connected to a mass-flow controller linked to the GC instrument. The gases in the reactor were purged at a rate of 15 sccm for 10 minutes to clean the GC lines before starting the GC measurement program. All compounds were quantified using external calibration curves generated with calibration standards. The amounts of propane, propylene and hydrogen in the gas phase were calculated from their partial pressures according to the ideal gas law (PV=nRT). The amounts of those gases dissolved in the solution were calculated based on Henry's Law (P=K_{H} χ), where Henry's law constants K_{H} of propane and propylene were determined by measuring their solubilities in acetonitrile under different partial pressures. The K_{H} of hydrogen was referred to a previous report, which is 5450 bar in MeCN. The yield of propylene was calculated based on the total amount of propane and propylene related by yield %=n_{propylene}/(n_{propylene}+nₚᵣₒₚₐₙₑ+n_{byproducts})×100%. The yield of hydrogen was calculated via the same way.

Possible liquid byproducts were identified by ¹H nuclear magnetic resonance (NMR) spectra. ¹H-NMR spectra were recorded on Varian® NMR instruments (either 500 or 600 MHz). Chemical shifts were illustrated in ppm using tetramethylsilane (TMS, 1 v/v % in CD₃CN) as an internal standard. Typically, 500 μL of a sample solution after catalysis was mixed with 100 μL of CD₃CN.

### Procedures for PDH Reactions

Catalytic PDH reactions were conducted in a glass pressure reaction vessel (3 oz or 85 mL in total volume) purchased from Andrews® Glass with customized Swagelok reactor tops. In a typical PDH experiment, 191 mg of NaDT was first dissolved in 30 mL acetonitrile at 90° C. After complete dissolution, the solution was cooled down to room temperature, then 32 mg of COPC and 18 mg dmgH₂ were added to the solution, and 10 mL acetonitrile was further added to flush the chemicals adhere to the vessel wall into the solution. Then the reactor was sealed by the modified Swagelok reactor top with needle-valve adaptor and O-ring. Grease was applied at the edge of the reactor to assure the airtightness of the reaction system. The air inside the vessel and dissolved in the solution was removed with freeze-pump-thaw technique for three times in the dry ice/acetone bath in a cryogenic storage dewar. Afterwards, the vessel was filled with 1 atm propane and was irradiated by a 365 nm LED (Howsuper®, H6015-S-6868-LG-365nm) with stirring at 700 rpm. The reaction temperature was maintained at 22° C. with a water bath in a jacketed beaker that was connected to a water-cooled chiller.

Ten reactions were carried out to dehydrogenation propane. Their conditions and results were summarized in Table 1 below. One reaction was performed on ethane. A comparative reaction shown in Table 1 used 3 mol % of NaDT without using any co-catalyst.

The method of this invention achieved as high as 69% of conversion of propane to propylene under the tested conditions. In a mixed propane/propylene substrate, the percentage of propylene was pushed to 95%, indicating that the method of this invention can achieve 95% conversion or higher when NaDT and COPC were optimally regenerated.

TABLE 1
Summary of PDH reactions

| | Catalyst | T | P | C | S |
|---|---|---|---|---|---|
| # | Substrate | (° C.) | (atm) | (%) | (%) |
| 1 | 3 mol % NaDT/3 mol % COPC | 22 | 1 | 48 | 99 |
| | Propane | | | | |
| 2 | 3 mol % NaDT/3 mol % COPC | 22 | 1 | 69 | 99 |
| | Fresh COPC after 20 h | | | | |
| | Propane | | | | |
| 3 | 3 mol % TPPDT/3 mol % COPC | 22 | 1 | 38 | — |
| | Propane | | | | |
| 4 | 3 mol % NaDT/3 mol % CoCl₂/6 mol % | 22 | 1 | 32 | — |
| | dmgH₂ | | | | |
| | Propane | | | | |
| 5 | 3 mol % NaDT/3 mol % COBF | 22 | 1 | 35 | — |
| | Propane | | | | |
| 6 | 3 mol % NaDT/3 mol % COPC/3 mol % | 22 | 1 | 55 | — |
| | dmgH₂ | | | | |
| | Propane | | | | |
| 7 | 3 mol % NaDT/3 mol % COPC/6 mol % | 22 | 1 | 60 | — |
| | dmgH₂ | | | | |
| | Propane | | | | |
| 8 | 3 mol % NaDT/3 mol % COPC/9 mol % | 22 | 1 | 61 | — |
| | dmgH₂ | | | | |
| | Propane | | | | |
| 9 | 3 mol % NaDT/3 mol % COPC | 22 | 1 | 82* | — |
| | Propane/63.5% propylene | | | | |
| 10 | 3 mol % NaDT/3 mol % COPC | 22 | 1 | 95* | — |
| | Propane/90.9% propylene | | | | |
| 11 | DT/COPC | 22 | 1 | 27 | 99 |
| | ethane | | | | |
| Ref. | Comparative: 3 mol % NaDT | 22 | 1 | 1.2 | |
| | Propane | | | | |

*T: temperature; P: pressure; C: propane conversion to propylene; S: propylene selectivity;
*propylene percentage.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of preparing propylene comprising the step of irradiating propane in the presence of a catalyst containing a decatungstate salt and a co-catalyst, wherein the co-catalyst contains cobalt or nickel.

2. The method of claim 1, wherein the decatungstate salt is sodium decatungstate (NaDT) or tetraphenylphosphonium decatungstate (TPPDT).

3. The method of claim 2, wherein the decatungstate salt is NaDT present at a level of 0.001 mol % to 30 mol % relative to propane.

4. The method of claim 1, wherein the co-catalyst is cobaloxime pyridine chloride (COPC), Co(dmgH)₂, N,N′,N″,N″′-(tetrafluorodiborato)bis[μ-(2,3-butanedionedioximato)]-cobalt(II) dihydrate, nickel(II) trifluoromethanesulfonate, or nickel(II) tetrafluoroborate.

5. The method of claim 4, wherein the co-catalyst is COPC present at a level of 0.001 mol % to 20 mol % relative to propane.

6. The method of claim 1, wherein the decatungstate salt is sodium decatungstate (NaDT) present at a level of 2.8 mol % to 3.2 mol % and the cocatalyst is cobaloxime pyridine chloride (COPC) present at a level of 0.5 mol % to 3.2 mol %, both relative to propane.

7. The method of claim 1, wherein the catalyst further contains dimethylglyoxime at a level of 0.01 mol % to 20 mol % relative to propane.

8. The method of claim 1, wherein the irradiating step is performed in a solvent selected from the group consisting of acetonitrile, acetone, benzonitrile, acetonitrile-water, and chlorobenzene.

9. The method of claim 1, wherein the irradiating step is performed absent of a solvent.

10. The method of claim 1, wherein the irradiating step is performed at a temperature of −40° C. to 80° C.

11. The method of claim 1, wherein the irradiating step is performed at a pressure of 0.001 bars to 14 bars.

12. The method of claim 1, wherein the irradiating step utilizes a light having a wavelength of 200 nm to 410 nm.

13. The method of claim 1, wherein the irradiation step is performed for 1 minutes to 50 hours.

14. The method of claim 13, wherein the irradiation step is performed for 1 hour to 20 hours.

15. The method of claim 1, wherein the irradiation step is performed in a batch reactor or a flow reactor.

16. A catalyst comprising a decatungstate salt and a co-catalyst, wherein the decatungstate salt is sodium decatungstate (NaDT) or tetraphenylphosphonium decatungstate (TPPDT), and the co-catalyst is cobaloxime pyridine chloride.

17. A catalyst comprising tetraphenylphosphonium decatungstate (TPPDT).

18. The catalyst of claim 17, further comprising cobalt or nickel.

19. A method of dehydrogenating an alkane, comprising the step of irradiating the alkane in the presence of the catalyst of claim 16, wherein the alkane contains 2 to 20 carbon atoms.

20. The method of claim 19, wherein the alkane is ethane, propane, butane, pentane, hexane, heptane, octane, nonane, decane, cyclopentane, cyclohexane, cycloheptane, cyclooctane, cyclononane, or cyclodecane.
